(19) United States
(12) Patent Application Publication
(10) Pub. No.: US 2024/0325735 A1
(43) Pub. Date: Oct. 3, 2024

(54) SHOCKING ELECTRODES FOR IMPLANTABLE MEDICAL DEVICES AND METHODS OF PRODUCING THE SHOCKING ELECTRODES

(71) Applicant: Pacesetter, Inc., Sylmar, CA (US)

(72) Inventors: Bryan Teague, Los Angeles, CA (US); Dorab N. Sethna, Culver City, CA (US); Gopesh Tilvawala, IV, Los Angeles, CA (US); Xiangqun Chen, Santa Clarita, CA (US); Kavous Sahabi, Winnetka, CA (US); Erin Deifel, Sylmar, CA (US)

(21) Appl. No.: 18/615,149

(22) Filed: Mar. 25, 2024

Related U.S. Application Data
(60) Provisional application No. 63/493,215, filed on Mar. 30, 2023.

(51) Int. Cl. A61N 1/05; A61N 1/02; A61N 1/39

(52) U.S. Cl. CPC A61N 1/0563; A61N 1/025; A61N 1/3956

(57) ABSTRACT

Shocking electrodes for implantable medical devices may include a coiled conductor that has an oblong cross-sectional shape and is configured to deliver high-voltage shocks for defibrillation therapy. The coiled conductor includes an electrically conductive element that is helically wrapped and defines the oblong cross-sectional shape. The electrically conductive element is one of (i) a multi-filar ribbon wire that includes multiple strands disposed side-by-side along a length of the multi-filar ribbon wire, (ii) a micro-coil that includes a coiled strand, or (iii) a micro-cable that includes multiple interwoven strands along a length of the micro-cable.

### CROSS-REFERENCE TO RELATED APPLICATIONS

This application is a non-provisional conversion of, and claims priority to, U.S. Provisional Patent Application No. 63/493,215, which was filed Mar. 30, 2023, and the entire disclosure of which is incorporated herein by reference.

### FIELD

Embodiments of the present disclosure generally relate to implantable medical devices (IMDs) having one or more shocking electrodes designed to deliver electrical stimulation for defibrillation therapy. In one or more embodiments, the shocking electrodes may be designed for subcutaneous placement within a patient.

### BACKGROUND

Some IMDs include circuitry that monitors a patient's heart rhythm to detect arrythmias, such as ventricular tachycardia, ventricular fibrillation, and/or atrial fibrillation. In response to detecting an arrythmia, the same or a different IMD may deliver a powerful electrical shock to defibrillate the heart. For example, implantable cardioverter defibrillators (ICDs) are IMDs which include a battery-operated pulse generator that generates high voltage shocks and at least one lead extending from the generator to deliver the shocks. Some ICD leads are intra-cardiac and/or transvenous, such that the leads are introduced on or in heart tissue or in surrounding blood vessels. Some ICD systems are subcutaneous and deliver defibrillation therapy without any intra-cardiac, endovascular, or transvenous leads. The subcutaneous ICDs (S-ICD) include at least one lead extending from the generator. The subcutaneous lead is implanted below the skin but outside of the cardiac tissue and blood vessels. The subcutaneous lead may be implanted along an exterior side of the rib cage, or along an interior side of the rib cage (e.g., substernal, non-vascular lead target placement). The lead may be proximate to the sternum. S-ICD systems eliminate certain risks associated with transvenous and/or intra-cardiac implanted leads, such as infections and lead failures that may require surgical intervention.

Conventional leads that deliver shocks for defibrillation therapy have cylindrical shocking electrodes, such that the cross-sectional shapes are circular. The cylindrical shocking electrodes may include a conductive coil in which a metal wire is helically wrapped around the lead body. The cylindrical shocking electrodes may be relatively simple to manufacture by feeding the wire to a rotating cylindrical mandrel. The tension on the wire may remain relatively constant as the mandrel and the coil rotate relative to the feeder.

Increasing the surface area of the shocking electrode in contact with the patient tissue, or as presented to the reference (return) electrode in the shocking circuit, can desirably reduce the shocking impedance, resulting in a lower defibrillation threshold and therefore a lower energy burden demanded from the defibrillator device, or higher success rate for rescuing a patient in ventricular fibrillation. The reduced device energy demand may permit reducing the size of one or more of the IMD components, such as the pulse generator (e.g., canister) via a smaller battery and/or smaller high voltage capacitor. Thus, there may be advantages to producing flatter shocking electrodes that have oblong cross-sectional shapes rather than circular cross-sections. However, producing leads with such oblong shocking electrodes is more complex than producing conventional leads having cylindrical shocking electrodes.

As an example, winding a conductor supplied from a feeder around a rotating, non-cylindrical mandrel may cause the tension in the conductor to fluctuate. Some perimeter segments of the mandrel are closer to the feeder than other perimeter segments of the mandrel, which results in some lengths of the conductor being in greater tension than other lengths of the conductor. The variable tension may cause undesirable inherent stress and/or torsion within the shocking electrode. Another difficulty associated with producing oblong shocking electrodes may be retaining the oblong cross-sectional shape of the shocking electrodes once the shocking electrode is separated from the mandrel and/or other support structures used during the forming process. For example, an operator may not be able to simply flatten a cylindrical shocking electrode to form an oblong shocking electrode because the conductor may have a shape memory with residual tension. Upon removing the compressive force, the residual tension may cause the shocking electrode to resiliently return towards the cylindrical shape or another un-controlled or unpredictable, undesirable shape.

A need remains for IMD shocking electrodes that have oblong cross-sectional shapes to achieve reduced shocking impedance, and methods of simply, reliably, and consistently forming such shocking electrodes.

### SUMMARY

In accordance with an embodiment, a shocking electrode of a lead for an implantable medical device (IMD) is provided. The shocking electrode includes a coiled conductor that has an oblong cross-sectional shape and is configured to deliver high-voltage shocks for defibrillation therapy. The coiled conductor includes an electrically conductive element that is helically wrapped and defines the oblong cross-sectional shape. The electrically conductive element is one of (i) a multi-filar ribbon wire that includes multiple strands disposed side-by-side along a length of the multi-filar ribbon wire, (ii) a micro-coil that includes a coiled strand, or (iii) a micro-cable that includes multiple interwoven strands along a length of the micro-cable.

The electrically conductive element may be the micro-coil. The micro-coil defines micro-turns and macro-turns. The macro-turns may define the oblong cross-sectional shape of the coiled conductor. In another example, the electrically conductive element is the multi-filar ribbon wire. At least a first strand of the strands in the multi-filar ribbon wire may be less electrically conductive than at least a second strand of the strands in the multi-filar ribbon wire. The coiled conductor may include one or more structural strands interwoven with the multi-filar ribbon wire. The one or more structural strands may exert tension on the coiled conductor to retain the oblong cross-sectional shape. In another example, the electrically conductive element is the micro-cable. The multiple interwoven strands of the micro-cable may be micro-strands.

The shocking electrode may include a base structure surrounded by the electrically conductive element. The base structure may have the oblong cross-sectional shape. The electrically conductive element may be embedded into an outer surface of the base structure. The electrically conductive element may be disposed at least partially within helical grooves defined along an outer surface of the base structure. The base structure may be composed of an electrically insulative material. In an example, the shocking electrode may include an overmold material that covers at least a portion of the coiled conductor. The overmold material may retain the oblong cross-sectional shape of the coiled conductor.

In accordance with an embodiment, a method of forming a shocking electrode of a lead for an IMD is provided. The method includes helically wrapping an electrically conductive element to define a coiled conductor that has an oblong cross-sectional shape. The coiled conductor delivers high-voltage shocks for defibrillation therapy. The electrically conductive element is one of (i) a multi-filar ribbon wire that includes multiple strands disposed side-by-side along a length of the multi-filar ribbon wire, (ii) a micro-coil that includes a coiled strand, or (iii) a micro-cable that includes multiple interwoven strands along a length of the micro-cable.

In an example, the helically wrapping includes helically wrapping the electrically conductive element on a mandrel that has an oblong cross-sectional shape. The method may include applying an overmold material on an outer surface of the coiled conductor while the coiled conductor is on the mandrel. The method may include dissolving at least some of the overmold material while the coiled conductor is on the mandrel, and extracting the coiled conductor from the mandrel after dissolving at least some of the overmold material. Dissolving at least some of the overmold material may include performing a heat treatment operation, performing laser ablation, performing sand-blasting, performing dry-ice blasting, and/or applying a chemical solvent onto the overmold material.

In an example, helically wrapping the electrically conductive element may include helically wrapping the electrically conductive element in-situ on a base structure of the shocking electrode. The base structure may have the oblong cross-sectional shape. Helically wrapping the electrically conductive element may include locating the electrically conductive element within a helical groove defined along an outer surface of the base structure. In another example, helically wrapping the electrically conductive element includes helically wrapping the electrically conductive element on a mandrel that has a cylindrical shape. The method may include extracting the coiled conductor from the mandrel, and flattening the coiled conductor after removal from the mandrel to achieve the oblong cross-sectional shape.

In accordance with an embodiment. a shocking electrode of a lead for an IMD is provided. The shocking electrode includes a conductor that is electrically conductive and configured to deliver high-voltage shocks for defibrillation therapy. The conductor has an oblong cross-sectional shape. The conductor is a weave structure defined by multiple strands that are woven together. The strands that are woven together include a first type of strands and a second type of strands that has a lower electrical conductivity than the first type of strands.

In an example, the second type of strands apply tension in the weave structure to retain the oblong cross-sectional shape of the conductor. In an example, the conductor is hollow, and the shocking electrode includes a base structure disposed within an interior of the conductor. The base structure may be composed of an electrically insulative material.

### BRIEF DESCRIPTION OF THE DRAWINGS

FIG. 1 illustrates a graphical representation of an implantable medical device (IMD) that is configured to apply defibrillation therapy in accordance with embodiments herein.

FIG. 2A illustrates a racetrack cross-sectional shape of the shocking electrode according to a first embodiment.

FIG. 2B illustrates an elliptical cross-sectional shape of the shocking electrode according to a second embodiment.

FIG. 2C illustrates a rounded rectangular cross-sectional shape of the shocking electrode according to a third embodiment.

FIG. 2D illustrates an oval cross-sectional shape of the shocking electrode according to a fourth embodiment.

FIG. 3 shows a block diagram of an IMD that is configured to be implanted into a patient.

FIG. 4 illustrates a plan view of a lead according to an embodiment.

FIG. 5 is a side view of a distal portion of the lead shown in FIG. 4.

FIG. 6 illustrates a cross-sectional shape of a shocking electrode of the lead in FIG. 4.

FIG. 7 illustrates the conductor of the shocking electrode in FIG. 4 according to an embodiment

FIG. 8 illustrates a close-up view of a portion of a weave structure of the conductor according to an embodiment.

FIG. 9 illustrates a portion of the conductor of the shocking electrode in FIG. 4 according to another embodiment in which the conductor is coiled.

FIG. 10 illustrates a portion of the conductor of the shocking electrode in FIG. 4 according to another coiled embodiment.

FIG. 11 is a perspective view of a portion of a lead for an IMD that includes a shocking electrode according to an embodiment.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

Embodiments may be implemented in connection with one or more implantable medical devices (IMDs). Non-limiting examples of IMDs include one or more of neurostimulator devices, implantable leadless monitoring and/or therapy devices, catheters, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference.

Additionally or alternatively, the IMD may be a subcutaneous IMD (e.g., a S-ICD) that includes one or more structural and/or functional aspects of the device(s) described in U.S. application Ser. No. 17/804,041, titled “Method And Implantable Medical Device For Reducing Defibrillation Impedance” and filed May 25, 2022; U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” and filed May 7, 2018; U.S. application Ser. No. 15/973,249, titled “Single Site Implantation Methods For Medical Devices Having Multiple Leads”, filed May 7, 2018, which are hereby incorporated by reference in their entireties. Further, one or more combinations of IMDs may be utilized from the above incorporated patents and applications in accordance with embodiments herein.

Optionally, the IMD may be a catheter which is temporarily implanted in the patient, such as during a medical procedure. The catheter may be, for example, a cardiac electrophysiology mapping catheter.

All references, including publications, patent applications and patents, cited herein are hereby incorporated by reference to the same extent as if each reference were individually and specifically indicated to be incorporated by reference and were set forth in its entirety herein.

### Terms

The terms “cardiac activity signal”, “cardiac activity signals”, “CA signal” and “CA signals” (collectively “CA signals”) are used interchangeably throughout to refer to measured signals indicative of cardiac activity by a region or chamber of interest. For example, the CA signals may be indicative of impedance, electrical or mechanical activity by one or more chambers (e.g., left or right ventricle, left or right atrium) of the heart and/or by a local region within the heart (e.g., impedance, electrical or mechanical activity at the AV node, along the septal wall, within the left or right bundle branch, within the purkinje fibers). The cardiac activity may be normal/healthy or abnormal/arrhythmic. An example of CA signals includes EGM signals. Electrical-based CA signals refer to an analog or digital electrical signal recorded by two or more electrodes, where the electrical signals are indicative of cardiac activity. Heart sound (HS) based CA signals refer to signals output by a heart sound sensor such as an accelerometer, where the HS based CA signals are indicative of one or more of the S1, S2, S3 and/or S4 heart sounds. Impedance based CA signals refer to impedance measurements recorded along an impedance vector between two or more electrodes, where the impedance measurements are indicative of cardiac activity.

The terms “high-voltage shock” and “HV shock” refer to defibrillation stimulus delivered at an energy level sufficient to terminate a defibrillation episode in a heart.

The term “defibrillation threshold” and acronym “DFT” refer to a minimum amount of energy needed to be delivered in a high-voltage shock of defibrillation therapy in order to return a heart to a normal rhythm from a condition in which the heart is experiencing a fibrillation dysrhythmia episode.

The term “oblong” as used herein refers to elongated shapes that are longer in at least one dimension than another dimension, such that the oblong shapes are not circular/cylindrical or square/cubic. The longest dimension of a cross-sectional shape is referred to herein as a “major dimension,” and a shorter dimension of the cross-sectional shape is referred to as a “minor dimension.” The minor dimension may be perpendicular to the major dimension.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “subcutaneous” shall mean below the skin, but not intravenous. For example, a subcutaneous lead and/or electrode is not located in a chamber of the heart, in a vein on the heart, or in the lateral or posterior branches of the coronary sinus. A subcutaneous lead and/or electrode may be located between the skin and the rib cage, or within an intercostal area between two ribs of the rib cage. The rib cage collectively refers to the ribs, sternum, and thoracic vertebrae. Optionally, subcutaneous placement may include the substernal, extra-pericardial space defined between the undersurface of the rib cage and the pericardium or outer portion of the heart.

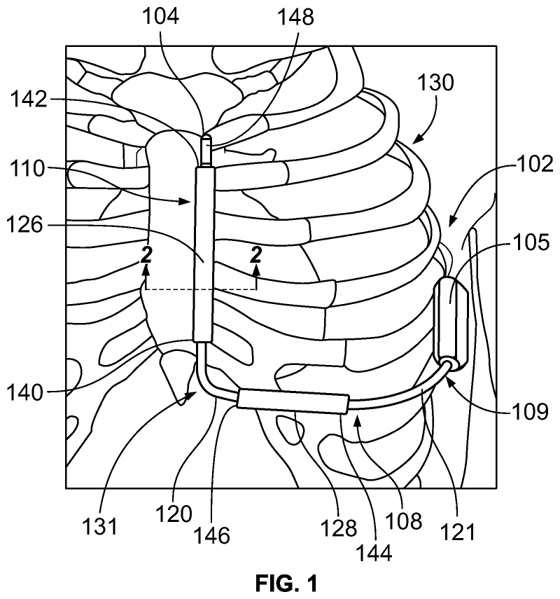

FIG. 1 illustrates a graphical representation of an implantable medical device (IMD) 102 that is configured to apply defibrillation therapy in accordance with embodiments herein. The IMD 102 in the illustrated embodiment is a subcutaneous implantable medical device (SIMD) that is configured to be implanted in a subcutaneous area exterior to the heart. The SIMD 102 includes a pulse generator 105 and at least one lead 120 that is operably coupled to the pulse generator 105. The “at least one lead” is hereinafter referred to as “the lead.” Nevertheless, it should be understood that the term, “the lead,” may mean only a single lead or may mean more than one single lead. The lead 120 includes a lead body 121 that is mechanically connected to the pulse generator 105 and extends from the pulse generator 105 to a distal tip 104 of the lead 120.

The pulse generator 105 includes a housing (e.g., canister) that contains power circuitry and energy storage devices for generating high-voltage shocks (HV shocks) for defibrillation therapy. The housing may be electrically conductive to form or constitute an electrode, referred to as the “CAN” electrode. The pulse generator 105 may be subcutaneously implanted within a pocket at a mid-axillary position along a portion of the rib cage 130 of the patient.

The lead 120 may be subcutaneously implanted. In particular embodiments, the SIMD 102 is an entirely or fully subcutaneous SIMD. The SIMD may not include a transvenous lead. The lead 120 in the illustrated embodiment includes a first or proximal segment 108 that extends from the pulse generator 105 along an inter-costal area between ribs. The lead 120 has a proximal end 109 that mechanically couples to the pulse generator 105, and electrically connects to the pulse generator 105 to establish conductive path(s) to the electrodes of the lead 120. The proximal segment 108 may be laterally oriented to extend along an anterior axillary area of the rib cage 130. The lead 120 has a second or distal segment 110 that extends from the proximal segment 108 to the distal tip 104. The distal segment 110 may extend along the sternum (e.g., over the sternum or parasternally within one to three centimeters from the sternum). The intersection between the distal and proximal segments 108, 110 may be located proximate to the xiphoid process of the patient.

The lead 120 includes at least one electrode that is electrically connected to the pulse generator 105 and delivers the HV shocks for defibrillation therapy. In the illustrated embodiment, the electrodes include a first or primary electrode 126 disposed along the distal segment 110 and a second or secondary electrode 128 disposed along the proximal segment 108. The electrodes 126, 128 are referred to herein as shocking electrodes. In an embodiment, when the pulse generator 105 generates a HV shock, the pulse generator 105 supplies electrical power to both of the shocking electrodes 126, 128, and both shocking electrodes 126, 128 may deliver the HV shocks based on the received electrical power. The shocking electrodes 126, 128 may concurrently deliver the HV shocks to different target areas of the heart.

The shocking electrodes 126, 128 are spaced apart from each other along the length of the lead 120 by a gap segment 131 of the lead body 121. The gap segment 131 may be proximate to the xiphoid process. The primary electrode 126 may be positioned along an anterior region of the chest, and the secondary electrode 128 may laterally extend between the primary electrode 126 and the pulse generator 105. The shocking electrodes 126, 128 may be subcutaneously positioned at a level that aligns with the heart of the patient for providing a sufficient amount of energy for defibrillation.

The primary electrode 126 may be oriented transverse to an orientation of the secondary electrode 128 when in the implanted position as shown in FIG. 1. For example, the primary electrode 126 has a first orientation extending from a proximal end 140 of the electrode 126 to a distal end 142 of the electrode 126 (defined along the length of the lead 120 relative to the pulse generator 105). The first orientation may be generally parallel to the midsternal line of the patient. The secondary electrode 128 has a second orientation extending from a proximal end 144 of the electrode 128 to a distal end 146 of the electrode 128. Optionally, the orientation of the secondary electrode 128 may define an angle between about 60 degrees and 120 degrees (e.g., such as 70 degrees to 110 degrees) relative to the orientation of the primary electrode 126. Due to the orientation, the lead 120 may be referred to as an L-shaped lead. The primary electrode 126 may be referred to as a parasternal electrode. The secondary electrode 128 may be referred to as a transverse electrode.

In an alternative embodiment, the SIMD 102 may lack the secondary electrode 128. For example, the proximal segment 108 may not have any shocking electrodes. The primary electrode 126 may be the only shocking electrode on the lead 120 that delivers the HV shocks supplied from the pulse generator 105.

In an embodiment, the lead 120 may include one or more sensing electrodes 148 to detect far field electrogram signals. The sensing electrode(s) 148 may collect subcutaneous cardiac activity (CA) signals in connection with multiple cardiac beats. In the illustrated embodiment, one sensing electrode 148 is disposed at the distal tip 104 of the lead 120. The SIMD 102 may process the CA signals to detect arrhythmias, such as ventricular tachycardia and/or atrial fibrillation. If an arrhythmia is detected, the SIMD 102 may automatically take one or more actions depending on characteristics of the arrythmia, such as type and severity. The actions may include delivering one or more electrical HV shocks (e.g., shock pulses) via the shocking electrodes 126, 128 in an attempt to achieve cardioversion. Optionally, another IMD may be implanted within the heart, such as a leadless pacemaker. The SIMD 102 may be configured to communicate with the other intra-cardiac IMD. For example, the intra-cardiac IMD may signal to the SIMD 102 when an arrythmia is detected for the SIMD 102 to deliver the HV shocks in response to receiving the signal.

In an embodiment, at least one of the shocking electrodes 126, 128 of the lead 120 has an oblong cross-sectional area, which may reduce the shocking impedance, as described in U.S. patent application Ser. No. 17/804,041. For example, relative to a cylindrical electrode, a flattened oblong shocking electrode may add significant tissue-contacting surface area through which electrical current can propagate in the subcutaneous space where the lead is placed. This increased surface area can reduce shocking impedance. A major dimension of the oblong shocking electrodes 126, 128 represents the largest or broadest dimension of the cross-sectional area. The cross-sectional area of the oblong shocking electrodes 126, 128 may include both the major dimension and a minor dimension that is perpendicular (i.e., orthogonal) to the major dimension. The minor dimension is smaller than the major dimension. The minor dimension may represent the smallest or narrowest dimension of the cross-sectional area, which is perpendicular to the major dimension. Optionally, an aspect ratio of the major dimension to the minor dimension may be at least 2:1. The oblong cross-sectional shape may be a racetrack shape with two opposite planar sides and two opposite curved sides between the two opposite planar sides. The shocking electrodes 126, 128 may have other oblong shapes, such as a rectangle with rounded corners, an oval (e.g., elliptical, egg-shaped, etc.), or the like. Optionally, the major dimension may be at least 10 F, which may achieve a low shocking impedance and maintain a low DFT, even in the presence of fat.

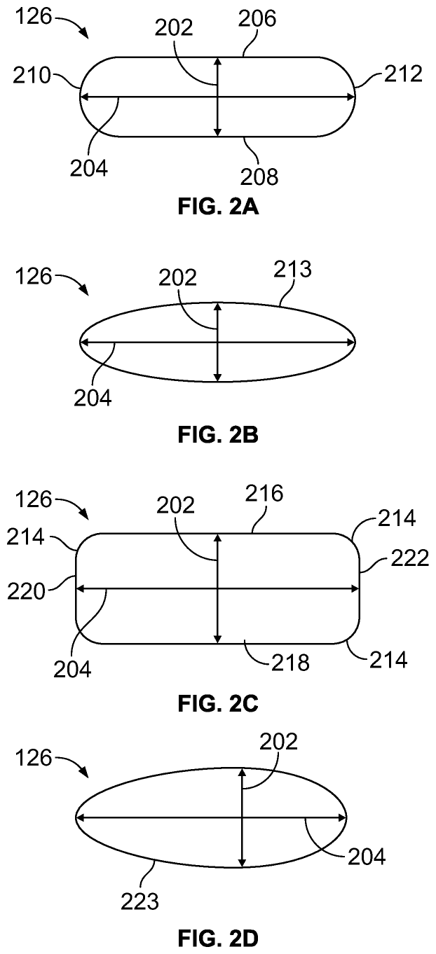

FIGS. 2A-2D illustrate different oblong cross-sectional shapes of the primary shocking electrode 126 of the lead 120 in FIG. 1 according to different embodiments. The cross-sections in FIGS. 2A-2D may be taken through line 2-2 in FIG. 1. The cross-sectional shapes are taken along a plane that is orthogonal to a tangential length direction of the lead 120 at the location of the cross-section. The illustrations in FIGS. 2A-D depict the perimeter shape (e.g., form) of the electrode 126 without showing the conductive elements and other features of the lead 120 and/or electrode 126 within the area defined by the perimeter shapes. Each of the oblong cross-sectional shapes in FIGS. 2A-2D has a respective minor dimension 202 and a respective major dimension 204. The minor dimension 202 may represent a thickness of the electrode 126, and the major dimension 204 represents a width of the electrode 126. The major dimension 204 is greater than the minor dimension 202.

FIG. 2A illustrates a racetrack cross-sectional shape of the shocking electrode according to a first embodiment. The shape of the shocking electrode 126 in FIG. 2A is referred to herein as a racetrack. The shocking electrode 126 has a first planar side 206 and a second planar side 208. The thickness of the electrode (e.g., the minor dimension 202) is defined between the first and second planar sides 206, 208. The planar sides 206, 208 may be parallel to each other. The electrode 126 has a first curved side 210 and a second curved side 212. Each of the curved sides 210, 212 extends from the first planar side 206 to the second planar side 208. The width of the electrode (e.g., the major dimension 204) is defined between the first and second curved sides 210, 212. In an embodiment, the curved sides 210, 212 have a radius of curvature that is half of the thickness of the electrode 126 (e.g., the diameter of the curvature is equal to the electrode thickness). The curved sides 210, 212 may have a different radius of curvature in other embodiments.

FIG. 2B illustrates an elliptical cross-sectional shape of the shocking electrode according to a second embodiment. The perimeter of the elliptical shocking electrode 126 has only curved sides 213; no planar surfaces. The perimeter may be traced by a point moving in a plane so that the sum of its distances from two focal points is constant.

FIG. 2C illustrates a rounded rectangular cross-sectional shape of the shocking electrode according to a third embodiment. The shocking electrode 126 is rectangular with rounded corners 214. For example, the shocking electrode 126 has two broad sides 216, 218 spaced apart from each other to define the thickness (e.g., minor dimension 202), and two narrow sides 220, 222 spaced apart from each other to define the width (e.g., major dimension 204). The broad sides 216, 218, and the narrow sides 220, 222 may be planar. The corners 214 are curved (e.g., rounded) to avoid snagging on patient tissue and/or implant tools, and/or to avoid injuring patient tissue.

FIG. 2D illustrates an oval cross-sectional shape of the shocking electrode according to a fourth embodiment. The oval shape in FIG. 2D is not an ellipse. Unlike the ellipse in FIG. 2B that has symmetry along both the minor dimension 202 and the major dimension 204, the oval shocking electrode 126 in FIG. 2D has symmetry only along the major dimension 204. The oval shocking electrode 126 has only curved sides 223.

The four oblong shapes are shown in FIGS. 2A-2D do not represent an exhaustive list of possible oblong cross-sectional shape for the shocking electrode 126. In one example, the electrode 126 may have a trapezoidal cross-sectional shape with rounded corners. Relative to the rectangular shape in FIG. 2C, the trapezoidal shape may be achieved by forming the first broad side 216 to be shorter than the opposite, second broad side 218, such that the narrow sides 220, 222 angle towards each other and are not parallel to each other.

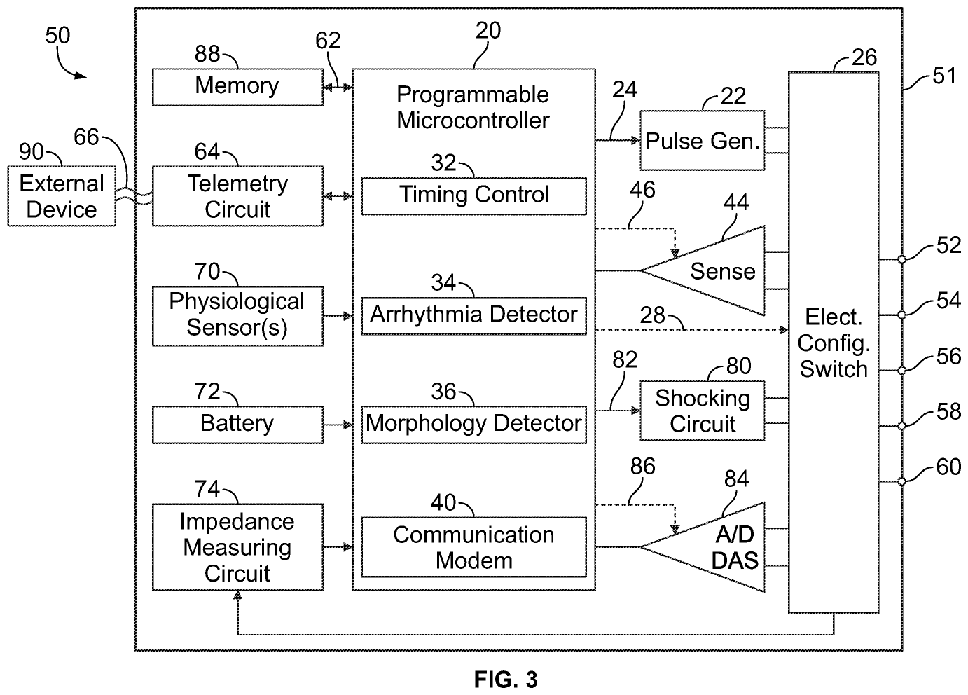

FIG. 3 shows a block diagram of an IMD 50 that is configured to be implanted into a patient. The IMD 50 may represent the SIMD 102 shown in FIG. 1. The IMD 50 may be implemented to monitor ventricular activity alone, or both ventricular and atrial activity through sensing circuit. The IMD 50 may treat both fast and slow arrhythmias with stimulation therapy, including cardioversion, pacing stimulation, an implantable cardioverter defibrillator, suspend tachycardia detection, tachyarrhythmia therapy, and/or the like.

The IMD 50 has a device case (or housing) 51 to hold the electronic/computing components. The case 51 (which can also be referred to as the “housing,” “can,” “encasing,” or “case electrode”) may be programmably selected to function as an electrode for certain sensing modes. Case 51 further includes a connector (not shown) with at least one terminal 52 and optionally additional terminals 54, 56, 58, 60. The terminals may be connected to electrodes that are located in various locations within and about the heart. The type and location of each electrode may vary. For example, the electrodes may include various combinations of ring, tip, coil, shocking electrodes, and the like.

The IMD 50 includes a programmable microcontroller 20 that controls various operations of the IMD 50, including cardiac monitoring and stimulation therapy. Microcontroller 20 includes a microprocessor (or equivalent control circuitry), RAM and/or ROM memory, logic and timing circuitry, state machine circuitry, and I/O circuitry. Microcontroller 20 includes an arrhythmia detector 34 that is configured to cardiac activity data to identify potential AF episodes as well as other arrhythmias (e.g., Tachycardias, Bradycardias, Asystole, etc.).

An electrode configuration switch 26 is optionally provided to allow selection of different electrode configurations under the control of the microcontroller 20. The electrode configuration switch 26 may include multiple switches for connecting the desired electrodes to the appropriate I/O circuits, thereby facilitating electrode programmability. The switch 26 is controlled by a control signal 28 from the microcontroller 20. Optionally, the switch 26 may be omitted and the I/O circuits directly connected to a housing electrode.

The IMD 50 further includes a chamber pulse generator 22 that generates stimulation pulses for connecting the desired electrodes to the appropriate I/O circuits, thereby facilitating electrode programmability. The pulse generator 22 is controlled by the microcontroller 20 via control signals 24. The IMD 50 includes a sensing circuit 44 selectively coupled to one or more electrodes that perform sensing operations through the switch 26 to detect cardiac activity. The sensing circuit 44 may include dedicated sense amplifiers, multiplexed amplifiers, or shared amplifiers. The sensing circuit 44 may operate in a unipolar sensing configuration or a bipolar sensing configuration. The output of the sensing circuit 44 is connected to the microcontroller 20 which, in turn, triggers, or inhibits the pulse generator 22 in response to the absence or presence of cardiac activity. The sensing circuit 44 receives a control signal 46 from the microcontroller 20 for purposes of controlling the gain, threshold, polarization, and timing of any blocking circuitry (not shown) coupled to the sensing circuit.

The IMD 50 further includes an analog-to-digital A/D data acquisition system (DAS) 84 coupled to one or more electrodes via the switch 26 to sample cardiac signals across any pair of desired electrodes. The A/D DAS 84 is controlled by a control signal 86 from the microcontroller 20.

The IMD 50 is communicatively connected to an external device 90. The external device 90 may communicate with a telemetry circuit 64 of the IMD 50 through a communication link 66. The external device 90 facilitates access by physicians to patient data as well as permitting the physician to review real-time cardiac signals while collected by the IMD 50.

The microcontroller 20 is coupled to a memory 88 by a suitable data/address bus 62. The memory 88 stores the programmable operating parameters used by the microcontroller 20 and/or data associated with the detection and determination of arrhythmias.

The IMD 50 may further include one or more physiologic sensors 70 adjust pacing stimulation rates, detect changes in cardiac output, changes in the physiological condition of the heart, and/or diurnal changes in activity (e.g., detecting sleep and wake states). Examples of physiological sensors 70 might include sensors that, for example, sense respiration rate, pH of blood, ventricular gradient, activity, body movement, position/posture, minute ventilation (MV), and/or the like.

The battery 72 provides operating power to all of the components in the IMD 50. The battery 72 is capable of operating at low current drains for long periods of time, and is capable of providing a high-current pulses (for capacitor charging) when the patient requires a shock pulse (e.g., in excess of 2 A, at voltages above 2 V, for periods of 10 seconds or more).

The IMD 50 further includes an impedance measuring circuit 74, which can be used for many things, including sensing respiration phase. The IMD 50 is further equipped with a communication modem (modulator/demodulator) 40 to enable wireless communication with the external device 90 and/or other external devices.

The IMD 50 includes a shocking circuit 80 controlled by control signals 82 generated by the microcontroller 20. The shocking circuit 80 generates shocking pulses of low (e.g., up to 0.5 joules), moderate (e.g., 0.5-10 joules), or high energy (e.g., 11 joules and above), as controlled by the microcontroller 20.

The microcontroller 20 may include other dedicated circuitry and/or firmware/software components, such as a timing control (module) 32 and a morphology detector (module) 36. The timing control 32 is used to control various timing parameters, such as stimulation pulses (e.g., pacing rate, atria-ventricular (AV) delay, atrial interconduction (A-A) delay, ventricular interconduction (V-V) delay, etc.) as well as to keep track of the timing of RR-intervals, refractory periods, blanking intervals, noise detection windows, evoked response windows, alert intervals, marker channel timing, and the like. The morphology detector 36 is configured to review and analyze one or more features of the morphology of cardiac activity signals, such as the morphology of detected R waves to determine whether to include or exclude one or more beats from further analysis.

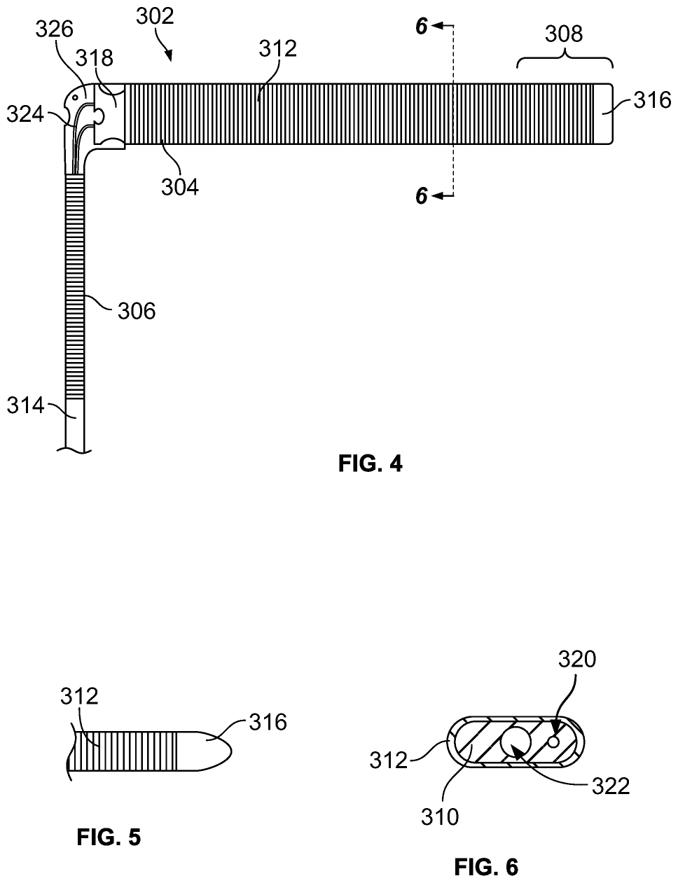

FIG. 4 illustrates a plan view of a lead 302 according to an embodiment. The lead 302 may be the lead 120 shown in FIG. 1. The lead 302 includes a primary shocking electrode 304 and a secondary shocking electrode 306. The primary shocking electrode 304 is located distal of the secondary shocking electrode 306 along the length of the lead 302. FIG. 5 is a side view of a distal portion 308 of the lead 302 shown in FIG. 4. FIG. 6 illustrates a cross-sectional shape of the primary shocking electrode 304 taken along line 6-6 in FIG. 4. The primary shocking electrode 304 and the secondary shocking electrode 306 optionally may represent the primary shocking electrode 126 and the secondary shocking electrode 128, respectively. In an embodiment, the primary shocking electrode 304 has an oblong cross-sectional shape, as shown in FIG. 6.

In an embodiment, the primary shocking electrode 304 (referred to herein simply as shocking electrode 304) includes an electrically insulative (e.g., dielectric) base structure 310 and a conductor 312 that surrounds the base structure 310. The base structure 310 may be composed of silicone rubber, polyurethane, and/or the like. The base structure 310 may be sufficiently flexible to enable the shocking electrode 304 to conform to the contour of the patient's body. Optionally, the base structure 310 may have the same or a similar composition as a lead body 314 of the lead 302. The lead 302 may include a sleeve 326 located between the two shocking electrodes 304, 306. The sleeve 326 may be a suture sleeve, strain relief sleeve, or the like. The sleeve 326 may have a defined curve to form an elbow segment that changes the angle of the lead 302. The lead 302 may include a distal sensing electrode 316 and a proximal sensing electrode 318 at either end of the shocking electrode 304.

The conductor 312 may be an electrically conductive metal material in the form of one or more strands (e.g., fibers, filars, filaments, wires, threads, etc.). The strand(s) of the conductor 312 may be wound, wrapped, wove, braided, or the like around the base structure 310. In an embodiment, the base structure 310 is unitary (e.g., one-piece, monolithic, etc.) and extends the entire length of the shocking electrode 304 between the sensing electrodes 316, 318. The base structure 310 and the conductor 312 may have oblong cross-sectional shapes. In the illustrated embodiment, both the base structure 310 and the conductor 312 have racetrack shapes.

In an embodiment, the base structure 310 defines one or more wire openings 320 for receiving electrical wire(s) 324 therethrough. The base structure 310 may define a lumen 322 for receiving the lead body of the lead 302, a rod of an implant tool, a flushing fluid, and/or the like. For example, the lead 302 may be self-implantable via blunt dissection by inserting an implant tool into the lumen 322 to manipulate and propel the shocking electrode 304 through the patient tissue during implant. The flushing fluid may be used to wet the interface between the base structure 310 and the conductor 312 and/or wet the electrode-tissue interface. The lumen 322 is optional, as the base structure 310 may lack the lumen 322 in another embodiment.

The oblong shocking electrode 304 may have various different constructions and may be produced by different processes according to the embodiments described herein. For example, the conductor 312 of the shocking electrode 304 can be constructed of a round or flat (solid) single-filar wire, a micro-coil, a multi-filar (round or ribbon) wire, a micro-cable including multiple woven filament strands, or the like. The conductor 312 may be assembled directly on the base structure 310 via helically wrapping the conductor 312 in a coil or weaving/braiding the conductor 312. In another embodiment, the conductor 312 may be wrapped or woven/braided on a mandrel that has an oblong cross-sectional shape. In still another embodiment, the conductor 312 may be wrapped or woven/braided to have a circular cross-section and then subsequently flattened via one or more post-processing tasks (to form the oblong cross-sectional shape) prior to loading the conductor 312 onto the base structure 310.

The shocking electrode 304 according to the embodiments described herein has the advantage of being highly flexible to be able to conform to the shape of the implant region and bend to permit a full range of patient movements after implantation without undue discomfort. In an embodiment, the implant region for the shocking electrode 304 may be the subcutaneous parasternal region of the patient (e.g., the location of the electrode 126 in FIG. 1). The added surface area of the non-circular shape helps decrease shocking impedance, which in turn reduces the required device energy burden, allowing for a smaller device (e.g., pulse generator). Small gaps between filars of the conductor 312 may accommodate tissue in-growth after implant, which helps to prevent migration of the lead/shocking electrode over time.

In various embodiments described herein, the process of forming the conductor 312 of the shocking electrode 304 to achieve and sustain the non-circular, oblong shape may include one or more of (i) heat treating the conductor 312, (ii) molding a dissolvable material onto the conductor 312, (iii) winding the conductor 312 into a highly flexible micro-coil structure, (iv) winding the conductor 312 on a non-circular, oblong mandrel, or (v) initially forming the conductor 312 on a circular mandrel and then flattening the conductor 312 to the desired oblong shape via one or more processes that cause the conductor 312 to maintain the flattened shape.

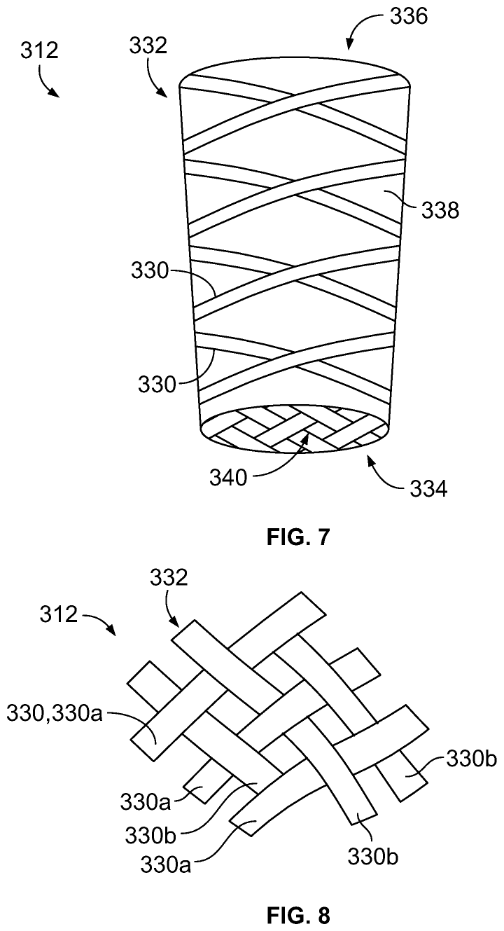

FIG. 7 illustrates the conductor 312 of the shocking electrode 304 in FIG. 4 according to an embodiment. In FIG. 7, the conductor 312 has multiple strands 330 that are interwoven to form a weave structure 332. The conductor 312 longitudinally extends from a proximal end 334 to a distal end 336. The conductor 312 has an oblong cross-sectional shape, along a plane that is perpendicular to a length axis of the conductor 312 from the proximal end 334 to the distal end 336. The oblong cross-sectional shape in the illustrated embodiment is elliptical, but may be another oblong shape in another embodiment.

The strands 330 in the weave structure 332 may have a cross-hatch pattern. For example, a first set of the strands 330 may be helically wrapped in a first wrapping angle. A second set of the strands 330 may be helically wrapped in a second wrapping angle that is different from the first wrapping angle such that the strands 330 in the second set interlace with the strands 330 in the first set. The weave structure 332 optionally may be a braided structure.

FIG. 8 illustrates a close-up view of a portion of the weave structure 332 of the conductor 312 according to an embodiment. The conductor 312 may include at least two different types of strands 330. For example, a first type of strand 330a may be electrically conductive to receive electrical power from the pulse generator 105 and emit energy in the form of shocking pulses for defibrillation therapy. The first type of strand 330a may be composed of an electrically conductive material that is safe for human tissue interaction. The strands 330a may include one or more metals, such as titanium, nickel, chromium, cobalt, stainless steel, MP35N, platinum, platinum-iridium alloy, tantalum, and/or the like.

In an embodiment, a second type of strand 330b in the conductor 312 may have less electrical conductivity than the first type. The strands 330b may be electrically insulative (e.g., dielectric). The strands 330b (referred to herein as insulative strands) may have greater elasticity properties than the strands 330a (referred to herein as conductive strands). In an example, the insulative strands 330b may be composed of nylon, polyester, PTFE, and/or the like. The insulative strands 330b may be used, at least in part, for shaping the conductor 312 and retaining the oblong cross-sectional shape. For example, the insulative strands 330b may be tightened to apply a tension on the conductive strands 330a. The tension supplied by the insulative strands 330b may withstand residual bias forces exerted by the conductive strands 330a to transition from the flattened, oblong shape to a cylindrical shape.

In the illustrative example, the insulative strands 330b are wrapped/woven in a first helical direction and the conductive strands 330a are wrapped/woven in a second helical direction. In an alternative embodiment, the strands 330a, 330b may be intermixed such that both types of strands 330a, 330b are helically wrapped/woven in the first and second helical directions. The weave structure 332 shown in FIG. 7 alternatively may be composed of only electrically conductive strands 330a.

The conductor 312 shown in FIG. 7 may be formed via various processes. In a first example, the strands 330 may be woven and/or braided directly onto a mandrel having an oblong cross-sectional shape. For example, the mandrel may have an elliptical cross-section. Once the weave structure 332 is complete (or mostly complete), the conductor 312 may be removed from the mandrel. At that point, the conductor 312 may be hollow, as shown in FIG. 7. The hollow conductor 312 may then be loaded onto the base structure 310 (shown in FIG. 6) such that the base structure 310 is received into a channel 340 defined by the hollow conductor 312. Alternatively, the hollow conductor 312 may be loaded onto a length of the lead body 314, rather than a discrete base structure.

Optionally, while the conductor 312 is disposed on the oblong mandrel, an overmold material may be applied to an outer (e.g., exterior) surface 338 of the conductor 312. The overmold material may be selected as a material that has a lower melting point than the conductor 312 (e.g., the strands 330). The overmold material may be a polymer, such as a thermoplastic. The overmold material may encase the conductor 312, like a cast, in the oblong shape of the mandrel. The process may subsequently include removing the overmold material. For example, a heat treatment (e.g., annealing) may be performed on the conductor 312 while on the mandrel. The heat treatment may relieve residual stresses within the strands 330 and dissolve/melt the overmold material. After the heat treatment, the conductor 312 may lack the overmold material. The conductor 312 may then be extracted from the mandrel, and the weave structure 332 may retain the oblong cross-sectional shape. Alternately, the overmold material may be removed in post-process via laser ablation, sand-blasting, dry-ice blasting, dissolving via chemical solvent, or the like. For example, the chemical solvent may be selected and/or applied to target the overmold material without interacting with the weave structure. The chemical solvent may dissolve the overmold material without damaging the weave structure. A complete removal of the restraining overmold may be performed. Alternately, a partial removal in discrete, pre-determined sections may be performed in order to permanently retain mechanical constraint of the conductor 312 in the discrete sections along the conductor's length.

In a second example formation process, the strands 330 may be woven/braided directly on the oblong base structure 310 shown in FIG. 6, rather than on a mandrel. As such, the weave structure 332 is formed in-situ on the base structure 310. In a third example formation process, the conductor 312 is initially formed in a cylindrical shape, with a circular cross-section. For example, the strands 330 may be woven/braided on a cylindrical mandrel and then removed from the mandrel. After forming the cylindrical weave structure 332, the conductor 312 may be compressed to flatten the conductor 312 into the oblong shape. For example, the conductor 312 may be compressed in a vice or clamp device. In an embodiment in which the weave structure 332 includes insulative strands 330b, the insulative strands 330b may be tightened to increase the tension and retain the oblong shape of the weave structure 332. Optionally, an overmold material may be applied onto the outer surface 338. When the overmold material hardens, the overmold material may retain the oblong shape of the weave structure 332. For example, the overmold material in this example may be an adhesive, an epoxy, or the like. Removal or management of the overmold may be considered as described above in the preceding paragraph.

In another embodiment, the second set of strands may be electrically conductive like the first set of strands, while providing the counter tension to maintain the shape of the conductor 312.

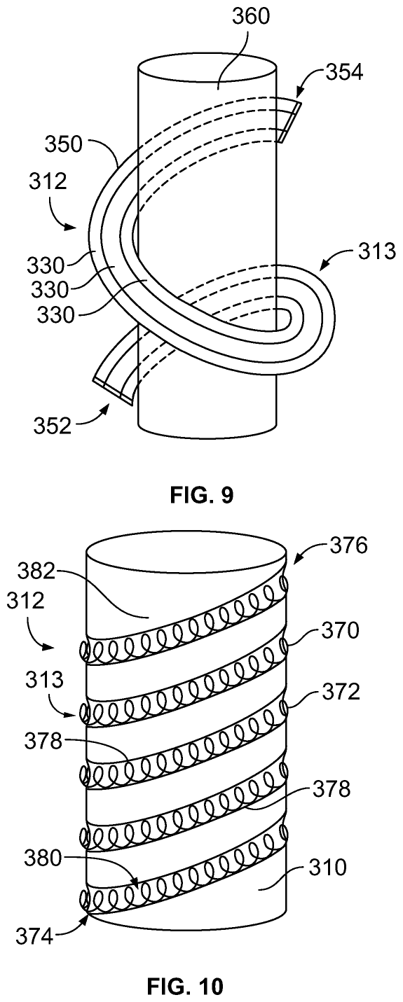

In some embodiments, the conductor 312 of the shocking electrode 304 is a coiled conductor 312. The coiled conductor 312 is defined by an electrically conductive element that is helically wrapped. The electrically conductive element defines the oblong cross-sectional shape of the coiled conductor 312. For example, FIG. 9 illustrates a portion of a coiled conductor 312 of the shocking electrode 304 according to an embodiment. In the illustrated embodiment, the electrically conductive element 313 is a multi-filar ribbon wire 350. The multi-filar ribbon wire 350 includes multiple strands 330 disposed side-by-side to collectively form a flat ribbon. The multi-filar ribbon wire 350 extends from a first end 352 to a second end 354 opposite the first end 352. Each of the strands 330 may extend from the first end 352 to the second end 354. At least some of the strands 330 in the multi-filar ribbon wire 350 are electrically conductive. FIG. 9 shows a length of the multi-filar ribbon wire 350 loosely wrapped along a portion of an oblong mandrel 360 for illustration, although the multi-filar ribbon wire 350 may be tightly wrapped with little or no gaps between turns when the conductor 312 is formed. The multi-filar ribbon wire 350 may have two or more strands 330 side-by-side. In the illustrated embodiment, the multi-filar ribbon wire 350 includes three strands 330. The strands 330 may be connected to one another at the ends 352, 354, and optionally along the length between the ends 352, 354. Forming the conductor 312 with the multi-filar ribbon wire 350 may reduce the length of the conductor 312 from the first end 352 to the second end 354, relative to wrapping just a single filar wire to form the conductor 312. The shorter length may contribute to a lower impedance. Optionally, a subset of one or more strands 330 in the multi-filar ribbon wire 350 may be insulative strands, and other strands 330 may be electrically conductive.

In another embodiment, the conductor 312 may include structural strands that are interwoven with the multi-filar ribbon wire 350. The structural strands may be wound in a first direction. The multi-filar ribbon wire 350 may be wound in a different, second direction and interwoven with the structural strands. The structural strands may be tightened to exert tension on the multi-filar ribbon wire 350 to retain the oblong shape of the conductor 312, as described with respect to the weave structure 332 in FIG. 8. In an example, the structural strands may be electrically insulative strands (e.g., formed of a dielectric material). In another example, the structural strands may be composed of an electrically conductive material. The structural strands may be discrete and separate from the strands 330 of the multi-filar ribbon wire 350.

FIG. 10 illustrates a portion of a coiled conductor 312 of the shocking electrode 304 according to another embodiment. In the illustrated embodiment, the electrically conductive element 313 is a micro-coil 370. The micro-coil 370 is helically wrapped to define the coiled conductor 312. The micro-coil 370 is itself a coiled wire. For example, the micro-coil 370 is a thin wire strand that has many micro-turns 372 (e.g., loops of the wire strand) along the length of the micro-coil 370 from a first end 374 to a second end 376 of the micro-coil 370 opposite the first end 374. In an example, the wire strand that forms the micro-coil 370 may be cylindrical (e.g., having a circular cross-section). A coil diameter of the micro-turns 372 may be on the order of the diameter of a single filar wire conventionally used as the helically-wrapped conductor of a shocking electrode. The diameter of a solid, single filar wire may range from 5 micrometers (μm) to 200 μm. The diameter of the strand of the micro-coil 370 may be significantly less than the diameter of the solid, single filar wire. In one example, the strand diameter of the micro-coil 370 may be approximately 25 μm. In another example, the strand diameter of the micro-coil 370 may be approximately one-tenth of the diameter of a single filar wire. The reduced strand diameter and micro-turns 372 may enable the micro-coil 370 to be more flexible than single and multi-filar wires. The micro-coil 370 would be inherently compliant to the base substrate over which it is wound, and would have less residual stress and shape memory than single and multi-filar wires as well, which desirably limits the propensity of the conductor 312 to spring-back from the oblong shape to a cylindrical shape.

The micro-coil 370 has multiple turns 378 (referred to as macro-turns to differentiate from the micro-turns 372) around an interior structure. In an embodiment, the interior structure is the base structure 310 of the shocking electrode 304. As such, the conductor 312 may be formed in-situ by wrapping the micro-coil 370 around the base structure 310. The spacing between the macro-turns 378 may be smaller than shown in FIG. 10 to increase the number of macro-turns 378. Optionally, the base structure 310 may define helical grooves 380 along an outer surface 382 thereof. The grooves 380 may be sized to accommodate the micro-coil 370 therein, to assist with the assembly process and ensure consistent spacing between the macro-turns 378. Alternatively, rather than define pre-formed grooves, the micro-coil 370 may be wrapped around the base structure 310 while the insulative material of the base structure 310 is in a green (e.g., softened or partially melted) state. The wrapping process may cause the micro-coil 370 to embed within the insulative material, which secures the micro-coil 370 in place on the base structure 310 upon the insulative material hardening. Another option may be to apply an overmold material over at least a portion of the micro-coil 370 that is wrapped around the base structure 310, where the overmold material assists with securing the micro-coil 370 to the base structure 310 and/or retaining the oblong shape of the conductor 312.

The spacings in and between the micro-turns 372 and the macro-turns 378 of the micro-coil 370 may enable growth of fibrotic tissue to prevent lead migration. The compliance and flexibility of the micro-coil 370 may enable the finished shocking electrode 304 to retain the desired oblong shape profile and to also be sufficiently flexible to easily bend along a full range of motion of the patient without causing discomfort.

In another embodiment, the electrically conductive element 313 is a micro-cable. The micro-cable is helically wrapped to define the coiled conductor 312. The micro-cable includes multiple interwoven strands. For example, the micro-cable may have a cable diameter that is approximately the same as the diameter of a conventional single filar wire. The strands of the micro-cable may be micro-strands. Each micro-strand may have a diameter that is approximately the same as that of the micro-coil strand diameter, which is substantially smaller than the single filar wire diameter. In an example, the micro-cable has between three and several hundred micro-strands that are woven or braided together to define the micro-cable. The thin strands may cause the micro-cable to be more flexible than solid single filar wires that have similar diameters as the micro-cable.

The various coiled conductors 312 shown and described herein may be formed via various processes. In a first example, the multi-filar ribbon wire 350, the micro-coil 370, and/or the micro-cable may be helically wound directly onto an oblong base structure 310, as shown in FIG. 10. In a second example, the multi-filar ribbon wire 350, the micro-coil 370, and/or the micro-cable may be helically wound directly onto a mandrel having an oblong cross-sectional shape, as shown in FIG. 9. Then, the conductor 312 is removed to achieve a hollow shape, which is then mounted to the base structure 310 (e.g., an insulative body, a sleeve, the lead body, or the like). Due to the oblong mandrel, the forming process may provide variable tension to the conductor 312 as it is wrapped. Optionally, while on the mandrel, a dissolvable overmold material may be applied on the outer surface of the conductor 312. Then, the assembly may be heated to dissolve the overmold material and relieve residual stress in the conductor 312 to maintain the oblong cross-sectional shape of the conductor 312 after extraction from the mandrel. In a third example, the multi-filar ribbon wire 350, the micro-coil 370, and/or the micro-cable may be initially helically wound in a cylindrical coil shape, and then subsequently compressed to form a flattened, oblong shape. The compression may cause some of the strands in the coil to slant at a different orientation than the orientation while the conductor was cylindrical.

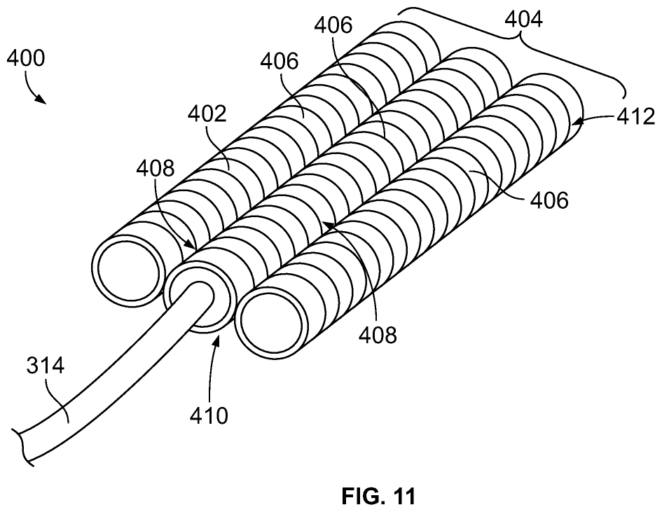

FIG. 11 is a perspective view of a portion of a lead 400 for an IMD that includes a shocking electrode 402 according to an embodiment. The lead 400 may be similar to the lead 302 shown in FIG. 4. The shocking electrode 402 may represent the shocking electrode 304. The shocking electrode 402 is mechanically and electrically connected to the lead body 314, which includes one or more electrical cables (e.g., wires) for providing electrical power to the shocking electrode 402 to supply high-voltage shocks for defibrillation therapy.

In the illustrated embodiment, the shocking electrode 402 is defined by a stack 404 of multiple cylindrical conductor elements 406. The cylindrical conductor elements 406 are arranged side-by-side, similar to the strands 330 of the multi-filar ribbon wire 350 in FIG. 9. For example, each conductor element 406 may extend a full length of the shocking electrode 402 between a proximal end 410 and a distal end 412 of the shocking electrode 402. The cylindrical conductor elements 406 are secured to each other at joints 408 between adjacent conductor elements 406. For example, the conductor elements 406 may be welded together at the joints 408. Optionally, each of the cylindrical conductor elements 406 may be a cylindrical coil conductor formed by helically wrapping a wire, such as a solid, single filar wire. Three cylindrical conductor elements 406 are shown in FIG. 11, but the shocking electrode 402 may have two, four, or more than four cylindrical conductor elements 406 in other embodiments. Although the constituent parts are each cylindrical, the shocking electrode 402 has an overall oblong cross-sectional shape. As such, the shocking electrode 402 may provide the electrical benefits of the oblong shocking electrodes described herein. Furthermore, the depressed areas at the joints 408 may provide space for fibrotic tissue in-growth to prevent lead migration.

The shocking electrode 402 may be formed by obtaining (e.g., retrieving or producing) multiple cylindrical conductor elements 406, then affixing the conductor elements 406 together side-by-side. The shocking electrode 402 may be electrically connected to the lead body 314 by electrically connected a power cable of the lead body 314 to one of the conductor elements 406. In the illustrated embodiment, the lead body 314 is mechanically connected to the middle conductor element 406 in the stack 404 of three conductor elements 406, but optionally may be coupled to a different conductor element 406. The other conductor elements 406 may be left as hollow or filled/plugged.

The shocking electrodes described with reference to FIGS. 4 through 11 can represent either or both of the shocking electrodes 126, 128 of the lead 120 shown in FIG. 1.

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage media having computer (device) readable program code embodied thereon.

Any combination of at least one non-signal computer (device) readable medium may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A shocking electrode of a lead for an implantable medical device (IMD), the shocking electrode comprising:
a coiled conductor that has an oblong cross-sectional shape and is configured to deliver high-voltage shocks for defibrillation therapy, wherein the coiled conductor includes an electrically conductive element that is helically wrapped and defines the oblong cross-sectional shape, wherein the electrically conductive element is one of (i) a multi-filar ribbon wire that includes multiple strands disposed side-by-side along a length of the multi-filar ribbon wire, (ii) a micro-coil that includes a coiled strand, or (iii) a micro-cable that includes multiple interwoven strands along a length of the micro-cable.

2. The shocking electrode of claim 1, wherein the electrically conductive element is the micro-coil, wherein the micro-coil defines micro-turns and macro-turns, and the macro-turns define the oblong cross-sectional shape of the coiled conductor.

3. The shocking electrode of claim 1, wherein the electrically conductive element is the multi-filar ribbon wire, wherein at least a first strand of the strands in the multi-filar ribbon wire is less electrically conductive than at least a second strand of the strands in the multi-filar ribbon wire.

4. The shocking electrode of claim 3, wherein the coiled conductor includes one or more structural strands interwoven with the multi-filar ribbon wire, the one or more structural strands configured to exert tension on the coiled conductor to retain the oblong cross-sectional shape.

5. The shocking electrode of claim 1, wherein the electrically conductive element is the micro-cable, and the multiple interwoven strands of the micro-cable are micro-strands.

6. The shocking electrode of claim 1, further comprising a base structure surrounded by the electrically conductive element, the base structure having the oblong cross-sectional shape.

7. The shocking electrode of claim 6, wherein the electrically conductive element is embedded into an outer surface of the base structure.

8. The shocking electrode of claim 6, wherein the electrically conductive element is disposed at least partially within helical grooves defined along an outer surface of the base structure.

9. The shocking electrode of claim 6, wherein the base structure is composed of an electrically insulative material.

10. The shocking electrode of claim 1, further comprising an overmold material that covers at least a portion of the coiled conductor, the overmold material configured to retain the oblong cross-sectional shape of the coiled conductor.

11. A method of forming a shocking electrode of a lead for an implantable medical device (IMD), the method comprising:
helically wrapping an electrically conductive element to define a coiled conductor that has an oblong cross-sectional shape, wherein the coiled conductor is configured to deliver high-voltage shocks for defibrillation therapy, wherein the electrically conductive element is one of (i) a multi-filar ribbon wire that includes multiple strands disposed side-by-side along a length of the multi-filar ribbon wire, (ii) a micro-coil that includes a coiled strand, or (iii) a micro-cable that includes multiple interwoven strands along a length of the micro-cable.

12. The method of claim 11, wherein the helically wrapping comprises helically wrapping the electrically conductive element on a mandrel that has an oblong cross-sectional shape.

13. The method of claim 12, further comprising:
applying an overmold material on an outer surface of the coiled conductor while the coiled conductor is on the mandrel;
dissolving at least some of the overmold material while the coiled conductor is on the mandrel; and
extracting the coiled conductor from the mandrel after the dissolving of at least some of the overmold material.

14. The method of claim 13, wherein dissolving the at least some of the overmold material comprises one or more of performing a heat treatment operation, performing laser ablation, performing sand-blasting, performing dry-ice blasting, or applying a chemical solvent onto the overmold material.

15. The method of claim 11, wherein helically wrapping the electrically conductive element comprises helically wrapping the electrically conductive element in-situ on a base structure of the shocking electrode, the base structure having the oblong cross-sectional shape.

16. The method of claim 15, wherein helically wrapping the electrically conductive element comprises locating the electrically conductive element within a helical groove defined along an outer surface of the base structure.

17. The method of claim 11, wherein helically wrapping the electrically conductive element comprises helically wrapping the electrically conductive element on a mandrel that has a cylindrical shape, wherein the method further comprises:
extracting the coiled conductor from the mandrel; and
flattening the coiled conductor after removal from the mandrel to achieve the oblong cross-sectional shape.

18. A shocking electrode of a lead for an implantable medical device (IMD), the shocking electrode comprising:
a conductor that is electrically conductive and configured to deliver high-voltage shocks for defibrillation therapy, the conductor having an oblong cross-sectional shape, wherein the conductor is a weave structure defined by multiple strands that are woven together,
wherein the strands that are woven together include a first type of strands and a second type of strands that has a lower electrical conductivity than the first type of strands.

19. The shocking electrode of claim 18, wherein the second type of strands apply tension in the weave structure to retain the oblong cross-sectional shape of the conductor.

20. The shocking electrode of claim 18, wherein the conductor is hollow and the shocking electrode further comprises a base structure disposed within an interior of the conductor, the base structure composed of an electrically insulative material.
